(19) United States
(12) Patent Application Publication
(10) Pub. No.: US 2021/0312836 A1
(43) Pub. Date: Oct. 7, 2021

(54) Blood Flow Assist Device

(71) Applicant: Fenwal, Inc., Lake Zurich, IL (US)

(72) Inventors: Brian C. Case, Lake Villa, IL (US); Kyungyoon Min, Kildeer, IL (US); Tammy Duncan, Spring Grove, IL (US)

(21) Appl. No.: 17/217,450

(22) Filed: Mar. 30, 2021

Related U.S. Application Data
(60) Provisional application No. 63/003,470, filed on Apr. 1, 2020.

(51) Int. Cl. G09B 23/30; G09B 23/32

(52) U.S. Cl. CPC G09B 23/303; A61B 5/15; G09B 23/32

(57) ABSTRACT

A blood flow assist device includes a flexible sleeve, at least one blood flow stimulator incorporated into the sleeve, and an actuator assembly coupled to the blood flow stimulator. The sleeve has a first portion configured to receive at least a portion of a arm and a second portion configured to engage a portion of a hand. The first and second portions of the sleeve properly position each blood flow stimulator on the arm. The actuator assembly individually controls and selectively actuates each blood flow stimulator to apply a stimulus to the arm to control blood flow through a vein in the arm or hand having a vein access device connected thereto. The blood flow assist device may be paired with a blood processing device, with a controller of the blood processing device controlling the actuator assembly based on a pressure and/or flow rate of blood into the blood processing device.

### CROSS-REFERENCE TO RELATED APPLICATIONS

This application claims the benefit of and priority of U.S. Provisional Patent Application Ser. No. 63/003,470, filed Apr. 1, 2020, the contents of which are incorporated by reference herein.

### BACKGROUND

### Field of the Disclosure

The present disclosure generally relates to systems, methods and devices for drawing blood from a patient or donor. More particularly, the present disclosure relates to a blood flow assist device, system, and method for ensuring proper blood flow from a patient or donor.

### Description of Related Art

Blood may be drawn from the arm or hand of a patient or donor for any of a number of reasons. During apheresis procedures, for example, blood is drawn from a patient or donor, a specific blood component is removed, and the remaining blood components are returned to the patient or donor. Apheresis procedures can be used to collect certain blood components (red blood cell, platelets, plasma, etc.) for donation, while also being useful in treating diseases. It is important during these procedures (and in any other procedure in which blood is drawn from a patient or donor) that there is proper blood flow. A low flow rate can result in a procedure taking a longer amount of time, while a high flow rate can result in vein collapse and/or may require operator interventions.

During these procedures, patients/donors may be required to squeeze their arm to ensure proper blood flow. Due to the length of the procedure, this squeezing can result in arm fatigue and general discomfort.

It is known to provide tourniquets or inflatable cuffs to increase blood flow through a patient or donor's arm during a blood collection procedure. For example, PCT Patent Application Publication No. WO 2019/006349 A1 (which is hereby incorporated herein by reference) discloses a system for increasing blood flow that includes an inflatable cuff that also contains a heating element.

### SUMMARY

There are several aspects of the present subject matter which may be embodied separately or together in the devices and systems described and claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto.

In one aspect, a blood flow assist device is provided. The device includes a flexible sleeve, at least one blood flow stimulator and an actuator assembly. The flexible sleeve has a first portion configured to receive at least a part of an arm of a donor or patient and a second portion configured to engage a portion of a hand of the patient or donor. The first and second portions of the flexible sleeve are configured to position the at least one blood flow stimulator at a target location of the arm. The at least one blood flow stimulator is incorporated into the flexible sleeve. The actuator assembly is coupled to at least one blood flow stimulator and configured to individually control and selectively actuate each blood flow stimulator to apply a stimulus to the arm to control blood flow through a vein in the arm or hand having a vein access device connected thereto.

In another aspect, there is provided a blood processing system. The blood processing system includes a blood flow assist device and a blood processing device. The blood flow assist device includes a flexible sleeve, a plurality of blood flow stimulators, and an actuator assembly. The flexible sleeve has a first portion configured to receive at least a part of an arm of a donor or patient and a second portion configured to engage a portion of a hand of the patient or donor. The plurality of blood flow stimulators are incorporated into the flexible sleeve with the first and second portions of the flexible sleeve being configured to position the at least one blood flow stimulator at a target location of the arm. The actuator assembly is configured to individually control and selectively actuate each blood flow stimulator to apply a stimulus to the arm to control blood flow through a vein in the arm or hand having a vein access device connected thereto. The blood processing device includes a controller, a blood draw pump configured to draw blood from the vein via the vein access device, and a pressure sensor. The controller is configured to receive a signal from the pressure sensor that is indicative of a capability of flow within the vein and to control operation of the blood draw pump and the actuator assembly based at least in part on said signal.

In another aspect, there is provided a method of controlling blood flow during a blood draw that includes associating a first portion of a flexible sleeve of a blood flow assist device to an arm of a patient or donor and a second portion of the flexible sleeve to a hand of the patient or donor, drawing blood from a vein of the arm, detecting a pressure at which the blood is being drawn from the arm or hand, and automatically applying a stimulus from the arm or hand using at least one blood flow stimulator of the blood flow assist device based at least in part on the pressure so as to modify a capability of flow within the vein, wherein the first and second portions of the flexible sleeve are configured to position the at least one blood flow stimulator at a target location of the arm.

### BRIEF DESCRIPTION OF THE DRAWINGS

FIG. 1 is a schematic view of an exemplary blood flow assist device of the present disclosure;

FIG. 2 is a top plan view of the blood flow assist device of FIG. 1; and

FIG. 3 is a schematic view of a blood processing system including a blood processing device and the blood flow assist device of FIGS. 1 and 2.

### DESCRIPTION OF THE ILLUSTRATED EMBODIMENTS

The embodiments disclosed herein are for the purpose of providing a description of the present subject matter, and it is understood that the subject matter may be embodied in various other forms and combinations not shown in detail. Therefore, specific designs and features disclosed herein are not to be interpreted as limiting the subject matter as defined in the accompanying claims.

While embodiments are described below in terms of use in an apheresis procedure, it will be appreciated that the devices, systems disclosed herein may be used in other types of blood collection or therapeutic procedures. Additionally, while the embodiments are described in terms of a sleeve that is worn around the arm of a patient or donor, the sleeve may alternatively be worn around other limbs of the user. Furthermore, the terms “patient” and “donor” are used interchangeably in the following description.

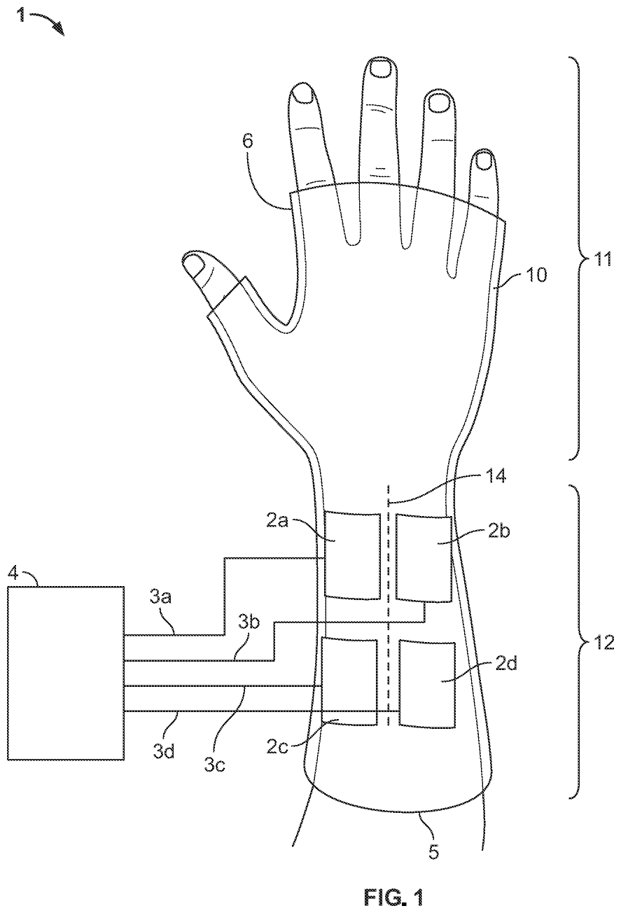

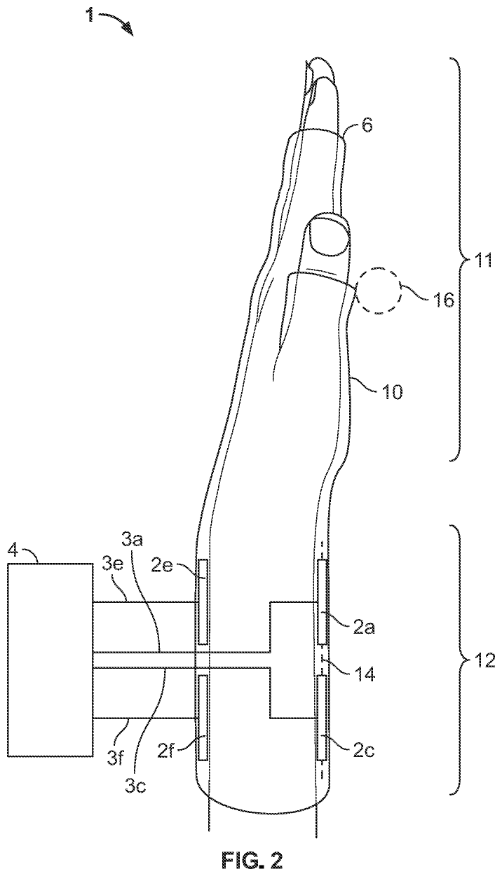

FIGS. 1 and 2 show an exemplary embodiment of a blood flow assist device 1 of the current disclosure. The blood flow assist device 1 includes a sleeve 10, which is sized and dimensioned to be placed around an arm or other limb. In the illustrated embodiment, sleeve 10 extends over and around a portion of the patient's arm near the site of a blood draw (which may comprise a needle or vein access device positioned proximally of the sleeve 10, such as in the crease of the arm or positioned distally of the sleeve 10, such as in the top of the hand).

The sleeve 10 may be variously configured without departing from the scope of the present disclosure. For example, the sleeve 10 may be comprised of one or more layers of a flexible material to accommodate different limb sizes. The material can include fabric made from synthetic or natural fibers, preferably material that is soft and hypoallergenic. The fibers can be elastic, for example. The fibers can be woven or nonwoven. Fibers can be comprised of polyamides, polyesters, polyurethanes, vinyl, polyethylene, cellulose, silk, wool, rayon, cotton, nylon and any other known fibers in the art. If non-woven, the fibers can be stapled, melt-blown, spun-bond, or any other known method of bonding. The fiber and fabric can be elastic and considered stretch fabric. The sleeve layer or layers can be comprised of blends or combinations of any of the above materials. Different layers of the sleeve 10 can be comprised of the same material or material combinations or different materials or material combinations.

The sleeve 10 may be impregnated or coated with an antimicrobial or other beneficial agent. Antimicrobial agents for medical applications, including, but not limited to, zinc, silver, and chlorhexidine compounds can be utilized.

The sleeve 10 may be disposable or reusable and may be sterilized or disinfected before use. If reusable, it can be cleaned, sterilized, and/or disinfected before each use.

In one embodiment, the sleeve 10 is a single piece that can be pulled on and off without manipulating a fastening or closure element, in which case it may be especially advantageous for the sleeve 10 to formed of a flexible, elastic material. Alternatively, the sleeve 10 may include a fastening or closure element, in which case it may be less important for the sleeve 10 to be formed of an elastic material, though flexibility may still be advantageous. Exemplary fastening or closure elements include hook-and-loop fasteners, buckles, adhesives, zippers, clasps, drawstrings, and the like.

Regardless of the material composition and accessories of the sleeve 10, at least one blood flow stimulator is incorporated into the sleeve 10. While it is within the scope of the present disclosure for a single blood flow stimulator to be employed, it may be advantageous for a plurality of smaller blood flow stimulators to be employed to provide more control over a stimulus applied to the limb by the sleeve 10, as will be described in greater detail herein. In the illustrated embodiment and by way of example only, eight flood flow stimulators are incorporated into the sleeve 10, with four of the blood flow stimulators 2a-2d being visible in FIG. 1. The other four blood flow stimulators are incorporated into other portions of the sleeve 10, with FIG. 2 showing the possible positions of two such blood flow stimulators 2e and 2f.

FIG. 2 shows the blood flow stimulators 2 embedded within or positioned between two layers of the sleeve 10, but it should be understood that a blood flow stimulator 2 may be otherwise incorporated into the sleeve 10. For example, in other embodiments, a blood flow stimulator 2 may be located on an inner surface of the sleeve 10 (i.e., configured to be placed into direct contact with a limb received within the sleeve 10) or on an outer surface of the sleeve 10. The preferred location of a blood flow stimulator 2 may depend on the nature of stimulus applied to the limb by the blood flow stimulator 2, with certain blood flow stimulators being best positioned in contact with a limb and others not requiring direct contact.

The blood flow stimulators 2 can be arranged in a variety of ways without departing from the scope of the present disclosure. In an exemplary embodiment, at least two blood flow stimulators 2 are associated with the same surface or side of the sleeve 10. In the illustrated embodiment, the sleeve 10 includes first and second surfaces (FIG. 2), with four blood flow stimulators 2a-2d associated with one of the surfaces (FIG. 1) and four blood flow stimulators (including blood flow stimulators 2e and 2f) associated with the other surface. As shown in FIG. 2, one of the surfaces of the sleeve 10 (along with the associated blood flow stimulators 2) is configured to be positioned against an anterior surface of a limb, while the opposite surface of the sleeve 10 (along with the associated blood flow stimulators 2) is positioned against a posterior surface of the limb. While the illustrated embodiment includes blood flow stimulators 2 positioned against opposing surfaces of a limb, it should be understood that blood flow stimulators 2 may be positioned at other locations, such as stretching across surfaces of the sleeve 10 and extending along the full circumference of the sleeve 10.

FIG. 1 shows an exemplary configuration in which four blood flow stimulators 2a-2d associated with one of the first and second surfaces of the sleeve 10 are arranged in a square or rectangular or grid pattern. The blood flow stimulators 2 associated with the opposing surface of the sleeve 10 may be arranged in a different pattern or in a similar, mirror image pattern. In one embodiment, the sleeve 10 may be configured for placement on either the left or right limb (as will be described in greater detail herein), in which case it may be advantageous to provide the two opposing surfaces of the sleeve 10 with blood flow stimulators 2 arranged in mirror image patterns.

Regardless of the particular configuration and arrangement of the blood flow stimulators 2, they are designed to apply a stimulus to a limb so as to control blood flow through a vein having a vein access device (e.g., a needle) inserted therein. An individual blood flow stimulator 2 may be controlled so as to apply a substantially uniform stimulus to a limb during a blood draw procedure. Alternatively, a blood flow stimulator 2 may instead be controlled to apply a varying stimulus to a limb during a blood draw procedure. For example, in the case of a procedure having multiple phases, a blood flow stimulator 2 may apply one stimulus during one phase and a different stimulus during another phase. This may include providing no stimulus during one of the phases.

The nature of the stimuli applied by the blood flow stimulators 2 may vary without departing from the scope of the present disclosure. In one embodiment, the blood flow stimulators 2 are configured to apply pressure to a limb so as to control blood flow through a vein of the limb. This may be achieved by blood flow stimulators 2 configured as inflatable bladders, for example. Inflatable bladders of the current disclosure can be comprised of a single layer or layers of different materials. Inflatable bladders can be comprised of a variety of materials, including thermoplastic materials and elastomers, for example. Suitable materials can include polyethylene, polypropylene, polyurethanes, polyamides, polyesters, ethylene vinyl acetate, natural or synthetic polyisoprene, polybutadiene, polychloroprene, silicone, nitrile rubbers, nylon, olefin, and polyvinyl chloride. The bladder layer or layers can be comprised of blends or combinations of any of the above materials. Different layers of the bladder or bladders can be comprised of the same material or material combinations or different materials or material combinations.

The bladder or bladders may be impregnated or coated with an antimicrobial or other beneficial agent, particularly if they are configured to be placed into direct contact with a limb. Antimicrobial agents for medical applications, including, but not limited to, zinc, silver, and chlorhexidine compounds can be utilized.

Each bladder can have an associated pressure sensor for providing feedback as to the current pressure of each bladder and controlling the pressure applied to the limb by the bladder.

Pressure may also be applied by a mechanical assembly, such as rollers. The rollers may move in a single direction or multiple directions to stimulate muscles of the limb so as to control blood flow through a vein of the limb. The rollers or other parts of a mechanical assembly may also vibrate in order to stimulate the muscles of the limb.

As an alternative to pressure, stimuli may instead take the form of electrical stimulations. Electrical stimulation devices, such as electrodes, can be incorporated into the sleeve 10 and selectively actuated to stimulate muscles of the limb so as to control blood flow through a vein of the limb.

In addition to blood flow stimulators 2, the sleeve 10 may additionally include at least one heating element 14 or a plurality of heating elements. The heating element(s) 14 may be composed of various materials, including, but not limited to metallic or ceramic materials. The heating element(s) 14 can be ribbons or wire. The heating element 14 is shown in dotted line in FIGS. 1 and 2 as a single heating element incorporated into the surface of the sleeve 10 positioned against the anterior surface of the limb, between the blood flow stimulators in a proximal section 12 of the sleeve 10. The heating element(s) can be arranged in various patterns across a section or entirety of the sleeve 10 and on either or both surfaces of the sleeve 10 positioned against the anterior and posterior surfaces of the limb.

An optional hand squeezing accessory 16 may also (or alternatively) be included as part of the sleeve 10 when the sleeve 10 is configured to be worn on an arm and hand. The hand squeezing accessory 16 may be a flexible material and may be formed into a ball or any other shape that is comfortably gripped or squeezed by hand. The optional hand squeezing accessory 16 is shown in FIG. 2 as a circular shape on a surface of the sleeve 10 in a distal section 11 of the sleeve 10. The hand squeezing accessory 16 can be attached by any conventional means and incorporated into or associated with the sleeve 10 at a suitable position (e.g., at a position overlaying the palm) so that it may be readily squeezed by the donor's hand during blood draw.

Actuation of the blood flow stimulators 2 may be controlled by an actuator assembly 4. For example, the blood flow stimulators 2 may be connected to the actuator assembly 4 by connectors 3. Each blood flow stimulator 2 can have its own connector 3, as shown in FIGS. 1 and 2, or multiple blood flow stimulators 2 can be connected to the actuator assembly 4 by a single connector 3. Actuator assembly 4 may also control any included heating element(s) 14.

The nature and configurations of the actuator assembly 4 and connectors 3 may vary, depending on the nature and configurations of the blood flow stimulators 2. For example, if the blood flow stimulators 2 are configured as inflatable bladders, the actuator assembly 4 may be configured as an air control manifold having a source of fluid and a drive mechanism (e.g., a compressor or pump). Alternatively, a separate drive mechanism may be provided (e.g., incorporated into a blood draw system) and coupled to the actuator assembly 4. The connectors 3 may be configured as tubes formed of any of a variety of materials, including a polymeric material, such as a plastic or rubber polymer, polyvinyl chloride, polyethylene, or a combination of these. A valving arrangement of the actuator assembly 4 would direct inflating fluid (e.g., air or a liquid) into and out of the various bladders, allowing for individual control and actuation of the bladders.

If the blood flow stimulators are configured as a mechanical assembly, such as rollers, the actuator assembly 4 may be configured with various components such as a screw, cam, rod, spring and/or shaft and may be used in combination with a motor. A controller of the actuator assembly 4 may direct various components to move in combination to produce a rolling or vibrating motion.

On the other hand, if the blood flow stimulators 2 are configured as electrical stimulation devices or electrodes, the actuator assembly 4 may be configured as a generator or power source or source of electrical stimulation, with the connectors 3 configured as conductive wiring. A controller of the actuator assembly 4 may direct current to the various electrodes, allowing for individual control and actuation of the electrodes.

One device or a combination of devices can perform the functions of the actuator assembly 4. The actuator assembly 4 may be configured to receive power from an external power source or may feature an on-board battery. The actuator assembly 4 is preferably configured to operate the blood flow stimulators 2 within safe limits based on pressure feedback from pressure sensors or electrical sensors.

The actuator assembly 4 is fixedly or removably attached to the sleeve 10, and may be located on the sleeve 10 or separate from the sleeve 10. The actuator assembly 4 (under the control of an internal or separate controller) controls the actuation and timing of the blood flow stimulators 2. The blood flow stimulators 2 can be stimulated simultaneously or in succession. In one embodiment, a distally positioned blood flow stimulator 2 is actuated, followed by the sequential actuation of blood flow stimulators 2 each positioned proximally of the previously actuated blood flow stimulator 2. Such a sequence simulates the muscle response when a first is being made to increase or enhance the blood flow in the vein. If blood flow stimulators 2 are positioned against both the anterior and posterior sides of the limb, the blood flow stimulators 2 on both sides of the limb may be actuated in such a sequence or the blood flow stimulators 2 on only one side of the limb (e.g., on the anterior side of the arm) may be actuated.

Properly stimulating a limb to control blood flow depends upon proper location of the various blood flow stimulators 2 with respect to the limb, to ensure that each blood flow stimulator 2 is applying a stimulus at a suitable location. To that end, the sleeve 10 may include a distal section 11 configured to be associated to a more distal portion or extremity 6 of the patient's limb and a proximal section 12 configured to be associated to a more proximal portion 5 of the patient's limb. In the illustrated embodiment, the distal section 11 of the sleeve 10 engages the hand, while the proximal section 12 surrounds a portion of the forearm or lower arm. In an alternative embodiment, the distal section 11 may be configured to engage a foot, while the proximal section 12 is configured to receive the lower leg or calf. The distal section 11 is configured to engage the extremity to prevent over-advancement or under-advancement of the sleeve 10 onto the limb. FIGS. 1 and 2, for example, show the distal section 11 of the sleeve 10 configured as a fingerless glove, which fits onto the hand in a particular position that determines the positions of the blood flow stimulators 2 with respect to the arm. As different patients have different limb lengths, differently sized sleeves 10 may be provided, such as a smaller, shorter sleeve for a child and a larger, longer sleeve for an adult patient.

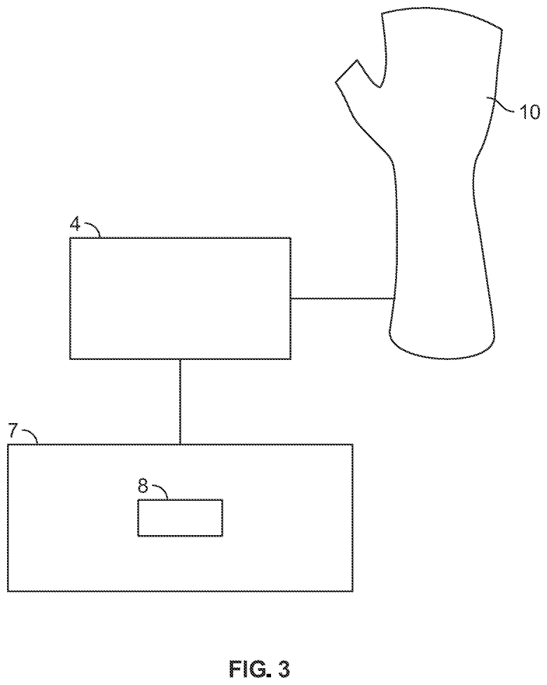

As described above, the actuator assembly 14 may be controlled by an internal or an external controller. FIG. 3 shows an arrangement in which the actuator assembly 4 is coupled to a blood draw device or system 7 (e.g., an apheresis device or system) having a controller 8 that controls operation of both the blood draw device 7 (or at least selected aspects thereof) and the actuator assembly 4. This connection can be wired or wireless. During blood draws, a needle or vein access device is placed into a vein of the patient's limb and is placed in fluid communication with the blood draw device 7. A blood draw pump of the blood draw device 7 is actuated by the controller 8 to draw blood into the blood draw device 7 from the vein, with pressure and/or flow rate being monitored and the controller 8 actuating the actuator assembly 4 and/or blood draw pump in response to pressure and/or flow rate levels.

When pressure is being monitored, the blood draw device 7 will include a pressure sensor (which may be variously configured without departing from the scope of the present disclosure) that monitors the input pressure of the blood draw pump, which is indicative of vein pressure and the capability of flow within the vein. When the input pressure is greater than a maximum or target pressure, the controller 8 will actuate the actuator assembly 4 to increase the stimulus applied in an attempt to increase the capability of flow within the vein and decrease the input pressure. The controller 8 may also generate an alert in the event of an elevated input pressure. The controller 8 may further control the operation of the blood draw pump based on the input pressure and/or an output pressure of the blood draw pump. For example, if the input pressure is above the target pressure, the controller 8 may command the blood draw pump to operate a lower rate in order to reduce the risk of vein collapse.

The hand squeezing accessory 16 can also be connected (wired or wirelessly) to the blood draw device or system 7. The blood draw device or system 7 can prompt the donor to squeeze the hand squeezing accessory when needed.

As for flow rate monitoring, either a direct blood flow sensor or an indirect approach may be employed. In one exemplary embodiment of an indirect approach, flow rate may be calculated based on the number of pump head rotations and the amount of fluid delivered to a particular receptacle or location (e.g., using a weight scale associated with a fluid container. If the draw rate is below a target rate or a target range, the controller 8 may control the actuator assembly 4 to stimulate the limb so as to increase the capability of flow within the vein. On the other hand, if the draw rate is above a target rate or a target range, the controller 8 may control the actuator assembly 4 to reduce or remove stimuli being applied to the limb. The blood draw device 7 may also monitor vein pressure and reduce or remove stimuli being applied to the limb to prevent vein collapse, as described above. In addition to a blood draw phase, a procedure executed by the blood draw device may have a variety of phases. In this case, the controller 8 and actuator assembly 4 can adjust the stimuli based on the phase of the procedure.

### Aspects

Aspect 1. A blood flow assist device comprising: a flexible sleeve with a first portion configured to receive at least a part of an arm of a patient or donor and a second portion configured to engage a portion of a hand of the patient or donor; at least one blood flow stimulator incorporated into the flexible sleeve; and an actuator assembly coupled to said at least one blood flow stimulator, wherein the actuator assembly is configured to individually control and selectively actuate each blood flow stimulator to apply a stimulus to the arm to control blood flow through a vein in the arm or hand having a vein access device connected thereto, and the first and second portions of the flexible sleeve are configured to position the at least one blood flow stimulator at a target location of the arm.

Aspect 2. The blood flow assist device of Aspect 1, wherein the actuator assembly is configured to control and actuate said at least one blood flow stimulator to apply stimuli to the arm of a patient or donor so as to simulate the condition of muscles in the arm when making a fist with a hand.

Aspect 3. The blood flow assist device of any one of the preceding Aspects, wherein the flexible sleeve includes opposing first and second surfaces configured to be positioned against opposing anterior and posterior surfaces of the arm when said at least a portion of the arm is received within the flexible sleeve, the at least one blood flow stimulator comprises a first plurality of blood flow stimulators incorporated into the first surface of the flexible sleeve and a second plurality of blood flow stimulators incorporated into the second surface of the flexible sleeve and coupled to the actuator assembly, the flexible sleeve is configured to receive the left arm of a patient or donor to position the first plurality of blood flow stimulators against the anterior surface of the left arm and the second plurality of blood flow stimulators against the posterior surface of the left arm, and the flexible sleeve is configured to receive the right arm of a patient or donor to position the second plurality of blood flow stimulators against the anterior surface of the right arm and the first plurality of blood flow stimulators against the posterior surface of the right arm.

Aspect 4. The blood flow assist device of Aspect 3, wherein the second plurality of blood flow stimulators are incorporated into the flexible sleeve in a pattern that is a mirror image of a pattern in which the first plurality of blood flow stimulators are incorporated into the flexible sleeve.

Aspect 5. The blood flow assist device of any one of Aspects 3-4, wherein the actuator assembly is configured to actuate each blood flow stimulator differently when said blood flow stimulator is positioned against the anterior surface of the arm and when said blood flow stimulator is positioned against the posterior surface of the arm.

Aspect 6. The blood flow assist device of any one of the preceding Aspects, wherein the actuator assembly is configured to actuate the at least one blood flow stimulator to apply a substantially uniform stimulus to the arm during a blood draw procedure.

Aspect 7. The blood flow assist device of any one of Aspects 1-5, wherein the actuator assembly is configured to actuate the at least one blood flow stimulator to apply a varying stimulus to the arm during a blood draw procedure.

Aspect 8. The blood flow assist device of any one of the preceding Aspects, wherein said at least one blood flow stimulator comprises a plurality of blood flow stimulators, and the actuator assembly is configured to actuate one of the blood flow stimulators to apply a different stimulus to the arm than another one of the blood flow stimulators during a blood draw procedure.

Aspect 9. The blood flow assist device of any one of the preceding Aspects, wherein the at least one blood flow stimulator comprises at least one inflatable bladder.

Aspect 10. The blood flow assist device of any one of Aspects 1-8, wherein the at least one blood flow stimulator comprises at least one electrode.

Aspect 11. The blood flow assist device of any one of the preceding Aspects, further comprising at least one heating element.

Aspect 12. The blood flow assist device of any one of the preceding Aspects, further comprising a hand squeezing accessory.

Aspect 13. A blood processing system, comprising: a blood flow assist device including a flexible sleeve with a first portion configured to receive at least a part of an arm of a patient or donor and a second portion configured to engage a portion of a hand of the patient or donor, at least one blood flow stimulator incorporated into the flexible sleeve, with the first and second portions of the flexible sleeve being configured to position the at least one blood flow stimulator at a target location of the arm, and an actuator assembly configured to individually control and selectively actuate each blood flow stimulator to apply a stimulus to the arm to control blood flow through a vein in the arm or hand having a vein access device connected thereto; and a blood processing device including a blood draw pump configured to draw blood from the vein via said vein access device, a pressure sensor, and a controller configured to receive a signal from the pressure sensor that is indicative of a capability of flow within the vein and to control operation of the blood draw pump and the actuator assembly based at least in part on said signal.

Aspect 14. The blood processing system of Aspect 13, wherein the controller is configured to compare the pressure at which blood is being drawn into the blood processing device from the arm or hand to a target pressure, and control the actuator assembly to increase the stimulus applied to the arm by at least one of the blood flow stimulators when the pressure at which blood is being drawn into the blood processing device from the arm or hand is greater than the target pressure.

Aspect 15. The blood processing system of Aspect 14, wherein the controller is configured to generate an alert when the pressure at which blood is being drawn into the blood processing device from the arm or hand is greater than the target pressure.

Aspect 16. The blood processing system of any one of Aspects 13-15, wherein the controller is configured to control the blood processing device to execute a procedure having a plurality of phases, control the actuator assembly to apply stimulus to the arm during one phase of the procedure, and control the actuator assembly to apply stimulus to the arm during another phase of the procedure that are different from the stimulus applied to the arm during said one phase of the procedure.

Aspect 17. The blood processing system of Aspect 16, wherein the controller is configured to control the actuator assembly to apply no stimulus to the arm during one of the phases of the procedure.

Aspect 18. The blood processing system of any one of Aspects 13-17, wherein the actuator assembly is configured to control and actuate said plurality of blood flow stimulators to apply stimulus to the arm so as to simulate the condition of muscles in the arm when making a fist.

Aspect 19. The blood processing system of any one of Aspects 13-18, wherein the flexible sleeve includes opposing first and second surfaces configured to be positioned against opposing anterior and posterior surfaces of the arm when said at least a portion of the arm is received within the flexible sleeve, the plurality of blood flow stimulators comprises a first plurality of blood flow stimulators incorporated into the first surface of the flexible sleeve and a second plurality of blood flow stimulators incorporated into the second surface of the flexible sleeve and coupled to the actuator assembly, the flexible sleeve is configured to receive the left arm of a patient or donor to position the first plurality of blood flow stimulators against the anterior surface of the left arm and the second plurality of blood flow stimulators against the posterior surface of the left arm, and the flexible sleeve is configured to receive the right arm of a patient or donor to position the second plurality of blood flow stimulators against the anterior surface of the right arm and the first plurality of blood flow stimulators against the posterior surface of the right arm.

Aspect 20. The blood processing system of Aspect 19, wherein the second plurality of blood flow stimulators are incorporated into the flexible sleeve in a pattern that is a mirror image of a pattern in which the first plurality of blood flow stimulators are incorporated into the flexible sleeve.

Aspect 21. The blood processing system of any one of Aspects 19-20, wherein the actuator assembly is configured to actuate each blood flow stimulator differently when said blood flow stimulator is positioned against the anterior surface of the arm and when said blood flow stimulator is positioned against the posterior surface of the arm.

Aspect 22. The blood processing system of any one of Aspects 13-21, wherein the actuator assembly is configured to control at least one of the blood flow stimulators to apply a substantially uniform stimulus to the arm during a blood draw procedure.

Aspect 23. The blood processing system of any one of Aspects 13-22, wherein the actuator assembly is configured to actuate at least one of the blood flow stimulators to apply a varying stimulus to the arm during a blood draw procedure.

Aspect 24. The blood processing system of any one of Aspects 13-23, wherein the actuator assembly is configured to actuate one of the blood flow stimulators to apply a different stimulus to the arm than another one of the blood flow stimulators during a blood draw procedure.

Aspect 25. The blood processing system of any one of Aspects 13-24, wherein the plurality of blood flow stimulators comprises a plurality of inflatable bladders.

Aspect 26. The blood processing system of any one of Aspects 13-24, wherein the plurality of blood flow stimulators comprises a plurality of electrodes.

Aspect 27. The blood processing system of any one of Aspects 13-26, wherein the blood flow assist device includes at least one heating element.

Aspect 28. The blood processing system of any one of Aspects 13-27, wherein the blood flow assist device includes a hand squeezing accessory.

Aspect 29. A method of controlling blood flow during a blood draw, comprising: associating a first portion of a flexible sleeve of a blood flow assist device to an arm of a patient or donor and a second portion of the flexible sleeve to a hand of the patient or donor; drawing blood from a vein of the arm or hand; detecting a pressure at which the blood is being drawn from the arm or hand; and automatically applying a stimulus to and/or removing a stimulus from the arm using at least one blood flow stimulator of the blood flow assist device based at least in part on the pressure so as to modify a capability of flow within the vein, wherein the first and second portions of the flexible sleeve are configured to position the at least one blood flow stimulator at a target location of the arm.

Aspect 30. The method of Aspect 29, wherein said automatically applying the stimulus to and/or removing the stimulus from the arm of the patient or donor based at least in part on the pressure so as to modify the capability of flow within the vein includes comparing the pressure to a target pressure, and automatically increasing at least one of the stimuli when the flow rate is greater than the target pressure.

Aspect 31. The method of Aspect 29, wherein said automatically applying the stimulus to and/or removing the stimulus from the arm of the patient or donor based at least in part on the pressure so as to modify the capability of flow within the vein includes generating an alert when the pressure is greater than the target pressure.

Aspect 32. The method of any one of Aspects 29-31, further comprising executing a procedure having a plurality of phases, automatically applying the stimulus to the arm during one phase of the procedure, and automatically applying a different stimulus to the arm during another phase of the procedure.

Aspect 33. The method of Aspect 32, further comprising applying no stimulus to the arm during one of the phases of the procedure.

Aspect 34. The method of any one of Aspects 29-33, wherein said automatically applying the stimulus to and/or removing the stimulus from the arm of the patient or donor based at least in part on the pressure so as to modify the capability of flow within the vein includes applying stimulus to the arm so as to simulate the condition of muscles in the arm when making a fist.

Aspect 35. The method of any one of Aspects 29-34, wherein said automatically applying the stimulus to and/or removing the stimulus from different locations of the arm of the patient or donor based at least in part on the pressure so as to modify the capability of flow within the vein includes applying stimulus to opposing anterior and posterior surfaces of the arm.

Aspect 36. The method of Aspect 35, wherein different stimuli are applied to the anterior and posterior surfaces of the arm.

Aspect 37. The method of any one of Aspects 29-36, wherein said automatically applying the stimulus to and/or removing the stimulus from the arm of the patient or donor based at least in part on the pressure so as to modify the capability of flow within the vein includes applying substantially uniform stimulus to the arm during a blood draw procedure.

Aspect 38. The method of any one of Aspects 29-36, wherein said automatically applying the stimulus to and/or removing the stimulus from the arm of the patient or donor based at least in part on the pressure so as to modify the capability of flow within the vein includes applying varying stimulus to the arm during a blood draw procedure.

Aspect 39. The method of any one of Aspects 29-38, wherein said automatically applying the stimulus to and/or removing the stimulus from the arm of the patient or donor based at least in part on the pressure so as to modify the capability of flow within the vein includes applying a stimulus to a first location on the arm and a different stimulus to a second location on the arm.

Aspect 40. The method of any one of Aspects 29-39, wherein said automatically applying the stimulus to and/or removing the stimulus from the arm of the patient or donor based at least in part on the pressure so as to modify the capability of flow within the vein includes applying pressure as a stimulus.

Aspect 41. The method of any one of Aspects 29-39, wherein said automatically applying the stimulus to and/or removing the stimulus from the arm of the patient or donor based at least in part on the pressure so as to modify the capability of flow within the vein includes applying electricity as a stimulus.

Aspect 42. The method of any one of Aspects 29-41, wherein the flexible sleeve includes a plurality of blood flow stimulators incorporated into the flexible sleeve at different locations.

Aspect 43. The method of any one of Aspects 29-42, wherein the flexible sleeve includes at least one heating element.

Aspect 44. The method of any one of Aspects 29-43, wherein the flexible sleeve includes a hand squeezing accessory.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

1. A blood flow assist device, comprising:
a flexible sleeve with a first portion configured to receive at least a part of an arm of a patient or donor and a second portion configured to engage a portion of a hand of the patient or donor;
at least one blood flow stimulator incorporated into the flexible sleeve; and
an actuator assembly coupled to said at least one blood flow stimulator, wherein
the actuator assembly is configured to individually control and selectively actuate each blood flow stimulator to apply a stimulus to the arm to control blood flow through a vein in the arm or hand having a vein access device connected thereto, and
the first and second portions of the flexible sleeve are configured to position the at least one blood flow stimulator at a target location of the arm.

2. The blood flow assist device of claim 1, wherein the actuator assembly is configured to control and actuate said at least one blood flow stimulator to apply stimuli to the arm of a patient or donor so as to simulate the condition of muscles in the arm when making a first with the hand.

3. The blood flow assist device of claim 1, wherein
the flexible sleeve includes opposing first and second surfaces configured to be positioned against opposing anterior and posterior surfaces of the arm when said at least a portion of the arm is received within the flexible sleeve,
the at least one blood flow stimulator comprises a first plurality of blood flow stimulators incorporated into the first surface of the flexible sleeve and a second plurality of blood flow stimulators incorporated into the second surface of the flexible sleeve and coupled to the actuator assembly,
the flexible sleeve is configured to receive the left arm of a patient or donor to position the first plurality of blood flow stimulators against the anterior surface of the left arm and the second plurality of blood flow stimulators against the posterior surface of the left arm, and
the flexible sleeve is configured to receive the right arm of a patient or donor to position the second plurality of blood flow stimulators against the anterior surface of the right arm and the first plurality of blood flow stimulators against the posterior surface of the right arm.

4. The blood flow assist device of claim 3, wherein the second plurality of blood flow stimulators are incorporated into the flexible sleeve in a pattern that is a mirror image of a pattern in which the first plurality of blood flow stimulators are incorporated into the flexible sleeve.

5. The blood flow assist device of claim 3, wherein the actuator assembly is configured to actuate each blood flow stimulator differently when said blood flow stimulator is positioned against the anterior surface of the arm and when said blood flow stimulator is positioned against the posterior surface of the arm.

6. The blood flow assist device of claim 1, wherein the actuator assembly is configured to actuate the at least one blood flow stimulator to apply a substantially uniform stimulus to the arm during a blood draw procedure.

7. The blood flow assist device of claim 1, wherein the actuator assembly is configured to actuate the at least one blood flow stimulator to apply a varying stimulus to the arm during a blood draw procedure.

8. The blood flow assist device of claim 1, wherein
said at least one blood flow stimulator comprises a plurality of blood flow stimulators, and
the actuator assembly is configured to actuate one of the blood flow stimulators to apply a different stimulus to the arm than another one of the blood flow stimulators during a blood draw procedure.

9. The blood flow assist device of claim 1, wherein the at least one blood flow stimulator comprises at least one inflatable bladder.

10. The blood flow assist device of claim 1, wherein the at least one blood flow stimulator comprises at least one electrode.

11. The blood flow assist device of claim 1, further comprising at least one heating element.

12. The blood flow assist device of claim 1, further comprising a hand squeezing accessory.

13. A blood processing system, comprising:
a blood flow assist device including
a flexible sleeve with a first portion configured to receive at least a part of an arm of a patient or donor and a second portion configured to engage a portion of a hand of the patient or donor,
at least one blood flow stimulator incorporated into the flexible sleeve, with the first and second portions of the flexible sleeve being configured to position the at least one blood flow stimulator at a target location of the arm, and
an actuator assembly configured to individually control and selectively actuate each blood flow stimulator to apply a stimulus to the arm to control blood flow through a vein in the arm or hand having a vein access device connected thereto; and
a blood processing device including
a blood draw pump configured to draw blood from the vein via said vein access device,
a pressure sensor, and
a controller configured to receive a signal from the pressure sensor that is indicative of a capability of flow within the vein and to control operation of the blood draw pump and the actuator assembly based at least in part on said signal.

14. The blood processing system of claim 13, wherein the controller is configured to
compare the pressure at which blood is being drawn into the blood processing device from the arm or hand to a target pressure, and
control the actuator assembly to increase the stimulus applied to the arm by at least one of the blood flow stimulators when the pressure at which blood is being drawn into the blood processing device from the arm or hand is greater than the target pressure.

15. The blood processing system of claim 14, wherein the controller is configured to generate an alert when the pressure at which blood is being drawn into the blood processing device from the arm or hand is greater than the target pressure.

16. The blood processing system of claim 13, wherein the controller is configured to
control the blood processing device to execute a procedure having a plurality of phases,
control the actuator assembly to apply stimulus to the arm during one phase of the procedure, and
control the actuator assembly to apply stimulus to the arm during another phase of the procedure that are different from the stimulus applied to the arm during said one phase of the procedure.

17. The blood processing system of claim 16, wherein the controller is configured to control the actuator assembly to apply no stimulus to the arm during one of the phases of the procedure.

18. The blood processing system of claim 13, wherein the actuator assembly is configured to control and actuate said plurality of blood flow stimulators to apply stimulus to the arm so as to simulate the condition of muscles in the arm when making a fist.

19. The blood processing system of claim 13, wherein
the flexible sleeve includes opposing first and second surfaces configured to be positioned against opposing anterior and posterior surfaces of the arm when said at least a portion of the arm is received within the flexible sleeve,
the plurality of blood flow stimulators comprises a first plurality of blood flow stimulators incorporated into the first surface of the flexible sleeve and a second plurality of blood flow stimulators incorporated into the second surface of the flexible sleeve and coupled to the actuator assembly,
the flexible sleeve is configured to receive the left arm of a patient or donor to position the first plurality of blood flow stimulators against the anterior surface of the left arm and the second plurality of blood flow stimulators against the posterior surface of the left arm, and
the flexible sleeve is configured to receive the right arm of a patient or donor to position the second plurality of blood flow stimulators against the anterior surface of the right arm and the first plurality of blood flow stimulators against the posterior surface of the right arm.

20. The blood processing system of claim 19, wherein the second plurality of blood flow stimulators are incorporated into the flexible sleeve in a pattern that is a mirror image of a pattern in which the first plurality of blood flow stimulators are incorporated into the flexible sleeve.

21. The blood processing system of claim 19, wherein the actuator assembly is configured to actuate each blood flow stimulator differently when said blood flow stimulator is positioned against the anterior surface of the arm and when said blood flow stimulator is positioned against the posterior surface of the arm.

22. The blood processing system of claim 13, wherein the actuator assembly is configured to control at least one of the blood flow stimulators to apply a substantially uniform stimulus to the arm during a blood draw procedure.

23. The blood processing system of claim 13, wherein the actuator assembly is configured to actuate at least one of the blood flow stimulators to apply a varying stimulus to the arm during a blood draw procedure.

24. The blood processing system of claim 13, wherein the actuator assembly is configured to actuate one of the blood flow stimulators to apply a different stimulus to the arm than another one of the blood flow stimulators during a blood draw procedure.

25. The blood processing system of claim 13, wherein the plurality of blood flow stimulators comprises a plurality of inflatable bladders.

26. The blood processing system of claim 13, wherein the plurality of blood flow stimulators comprises a plurality of electrodes.

27. The blood processing system of claim 13, wherein the blood flow assist device includes at least one heating element.

28. The blood processing system of claim 13, wherein the blood flow assist device includes a hand squeezing accessory.

29. A method of controlling blood flow during a blood draw, comprising:
associating a first portion of a flexible sleeve of a blood flow assist device to an arm of a patient or donor and a second portion of the flexible sleeve to a hand of the patient or donor;
drawing blood from a vein of the arm or hand;
detecting a pressure at which the blood is being drawn from the arm or hand; and
automatically applying a stimulus to and/or removing a stimulus from the arm using at least one blood flow stimulator of the blood flow assist device based at least in part on the pressure so as to modify a capability of flow within the vein, wherein the first and second portions of the flexible sleeve are configured to position the at least one blood flow stimulator at a target location of the arm.

30. The method of claim 29, wherein said automatically applying the stimulus to and/or removing the stimulus from the arm of the patient or donor based at least in part on the pressure so as to modify the capability of flow within the vein includes
comparing the pressure to a target pressure, and
automatically increasing at least one of the stimuli when the flow rate is greater than the target pressure.

31. The method of claim 29, wherein said automatically applying the stimulus to and/or removing the stimulus from the arm of the patient or donor based at least in part on the pressure so as to modify the capability of flow within the vein includes generating an alert when the pressure is greater than the target pressure.

32. The method of claim 29, further comprising
executing a procedure having a plurality of phases,
automatically applying the stimulus to the arm during one phase of the procedure, and
automatically applying a different stimulus to the arm during another phase of the procedure.

33. The method of claim 32, further comprising applying no stimulus to the arm during one of the phases of the procedure.

34. The method of claim 29, wherein said automatically applying the stimulus to and/or removing the stimulus from the arm of the patient or donor based at least in part on the pressure so as to modify the capability of flow within the vein includes applying stimulus to the arm so as to simulate the condition of muscles in the arm when making a fist.

35. The method of claim 29, wherein said automatically applying the stimulus to and/or removing the stimulus from different locations of the arm of the patient or donor based at least in part on the pressure so as to modify the capability of flow within the vein includes applying stimulus to opposing anterior and posterior surfaces of the arm.

36. The method of claim 35, wherein different stimuli are applied to the anterior and posterior surfaces of the arm.

37. The method of claim 29, wherein said automatically applying the stimulus to and/or removing the stimulus from the arm of the patient or donor based at least in part on the pressure so as to modify the capability of flow within the vein includes applying substantially uniform stimulus to the arm during a blood draw procedure.

38. The method of claim 29, wherein said automatically applying the stimulus to and/or removing the stimulus from the arm of the patient or donor based at least in part on the pressure so as to modify the capability of flow within the vein includes applying varying stimulus to the arm during a blood draw procedure.

39. The method of claim 29, wherein said automatically applying the stimulus to and/or removing the stimulus from the arm of the patient or donor based at least in part on the pressure so as to modify the capability of flow within the vein includes applying a stimulus to a first location on the arm and a different stimulus to a second location on the arm.

40. The method of claim 29, wherein said automatically applying the stimulus to and/or removing the stimulus from the arm of the patient or donor based at least in part on the pressure so as to modify the capability of flow within the vein includes applying pressure as a stimulus.

41. The method of claim 29, wherein said automatically applying the stimulus to and/or removing the stimulus from the arm of the patient or donor based at least in part on the pressure so as to modify the capability of flow within the vein includes applying electricity as a stimulus.

42. The method of claim 29, wherein the flexible sleeve includes a plurality of blood flow stimulators incorporated into the flexible sleeve at different locations.

43. The method of claim 29, wherein the flexible sleeve includes at least one heating element.

44. The method of claim 29, wherein the flexible sleeve includes a hand squeezing accessory.
